(19) United States
(12) Patent Application Publication
(10) Pub. No.: US 2026/0321428 A1
(43) Pub. Date: Oct. 15, 2026

(54) DUAL-DIRECTIONAL CONTENT GENERATION SYSTEM, METHOD AND COMPUTER READABLE STORAGE MEDIUM

(71) Applicant: WellnessMind AI Ltd, ANAHEIM, CA (US)

(72) Inventors: Xiaozhong Liu, Worcester, MA (US); Zhuoren Jiang, Hangzhou (CN)

(21) Appl. No.: 19/178,386

(22) Filed: Apr. 14, 2025

(51) Int. Cl. G16H 50/30; G06F 40/40; G16H 10/60; G16H 40/20; G16H 40/67; G16H 80/00

(52) U.S. Cl. CPC G16H 50/30; G16H 10/60; G16H 40/20; G16H 40/67; G16H 80/00; G06F 40/40

(57) ABSTRACT

Disclosed is a dual-directional content generation system, method and computer readable storage medium based on generative large model. By dynamically adapting to the needs of patients and healthcare professionals, the generative large language model is combined to generate the accurate and efficient personalized content for a patient and a doctor for health management, abnormality handling, and medication recommendation support.

### TECHNICAL FIELD

The present disclosure relates to a dual-directional content generation system, method and computer readable storage medium, based on generative large language model, applicable to the technical field of intelligent processing of medical information.

### BACKGROUND

Existing medical information generation systems are usually suitable to a single user type, such as a patient or a doctor, and thus lack flexibility and diversity, which are difficult to meet the needs of different users in medical scenarios simultaneously. Specifically, the current generation of patient health advice is mostly based on fixed templates or a single generative large language model, and the content cannot be dynamically adapted to the user's real-time health data, so the relevance of the advice is low. The generation of reports for doctors often relies on a structural data or simply spliced templates, which lacks in-depth study and integration of medical knowledge. The systems lack a unified framework to support the synchronous generation of patient health advice and doctor's diagnostic reports, which limits the quality and efficiency of content generation. In the case of health anomalies or emergencies, how to quickly and accurately generate content targeted at both patients and healthcare professionals, so as to help patients receive timely treatment, is an important issue to be solved.

### SUMMARY

The present disclosure relates to a dual-directional content generation system, method and computer readable storage medium based on generative large language model, which generates accurate and efficient personalized content for a patient and a doctor by dynamically adapting to the needs of patients and healthcare professionals, in conjunction with generative large language model, for health management, abnormality handling, and medical decision support.

The present disclosure relates to a dual-directional content generation system based on a generative large language model, comprising a detection and generation subsystem and a support and optimization subsystem, wherein the detection and generation subsystem comprises a multi-modal data collection and processing module, a health status evaluation module and a dual-directional content generation module, the support and optimization subsystem comprises a content classification and optimization module and an information update and feedback module; wherein, the multi-modal data collection and processing module integrates a collected physiological signal data and a subjective symptom textual description into a structured health data by means of a denoising algorithm and a time series modeling; the health status evaluation module evaluates a health status of a user by analyzing the structured health data in real time through a deep sequence model; the dual-directional content generation module dynamically generates personalized treatment recommendations adapted to user's needs and specialized diagnostic reports provided to a doctor based on health status assessment results; the content classification and optimization module adopts LORA fine-tuning technology to optimize the generated content, verifies and optimizes the generated content by a medical knowledge base and a regulatory rule base; the information update and feedback module combines user's health status and medical resource condition, dynamically adjusts a frequency of content generation, and provides personalized health reports and a follow-up appointment planning.

In certain embodiment, the content generated by the dual-directional content generation module comprises an emergency situation generated content and a non-emergency situation generated content. Preferably, the content classification and optimization module includes a training phase and an inference phase; the training phase is to optimize the generative large language model so as to generate an adapted content per different target users; the inference phase is to classify and adjust the generated content to ensure matching the needs of the target users.

Preferably, in the training phase, the data is classified and annotated per the target users to provide a direction for the training process; parameters of the generative large language model is optimized through fine-tuning technique to enable generating the adapted content for a patient and a doctor; training samples are extended through data augmentation to improve the model's ability to adapt to diverse scenarios.

Preferably, in the inference phase, the output is optimized based on the preliminary results of the generated content; the input information of the inference phase is the preliminary generated content of the dual-directional content generation module, and the output information is the final content that meets the needs of the target user.

In certain embodiment, the information update and feedback module comprises a training phase and an inference phase, the training phase is to add a new data to model training process by continuously collecting new data, dynamically adjust generation strategies and the parameters of the health status evaluation model, continuously update training dataset and optimize model performance; the inference phase is to collect latest health data and feedback information from the user and update the system database periodically or per triggering conditions to keep the evaluation model up-to-date; and to adjust the strategies of the health status evaluation model and the content generation module per the new data to optimize the output of the system.

Preferably, workflow of the system comprises:
(1) collecting and processing the data
wherein the user's physiological signals X and the subjective symptom textual description F provided by the user through an input device are collected in real-time; for the physiological signals data, a denoising process is performed to ensure the quality of the data by cleaning up the incomplete or abnormal signals; for the textual data, the text vectorization processing is performed; the pre-processed physiological signals X′ and subjective symptom textual description F′ are inputted to the health status evaluation module;
(2) evaluating and classifying health status
wherein pre-processed physiological signals X′ and subjective symptom textual description F′ are integrated into a multi-modal analysis model, where trend data of the physiological signals and textual representations of subjective symptoms are used as a basis for evaluation to comprehensively analyzing the data;
(3) generating dual-directional content
wherein content generation process for different users in different situations is triggered based on health status assessment results.

In certain embodiment, evaluating and classifying health status comprises: detecting sudden changes using time series modeling; classifying health status and determining whether current situation is an emergency situation, and ultimately output health state classification information that serves as a trigger for generating subsequent content.

In certain embodiment, the content generation process comprises the following steps:
(1) in data preprocessing phase, structuring multi-modal data of physiological signals and textual description, using as input data for the health status evaluation module and the dual-directional content generation module;
(2) evaluating and classifying user's health status by comprehensive analyzing and time series modeling the input data, for triggering of subsequent content generation modules;
(3) based on health status assessment results, generating a personalized content for a patient and a doctor respectively, and adjusting generation logic according to an emergency or a non-emergency situation to ensure that generated content is compliant and provides treatment recommendation.

Preferably, content classification and generation optimization process comprises a training phase and an inference phase, the training phase comprises low rank matrix LORA fine-tuning for different objects and annotates dataset for a patient and a doctor respectively by a two-channel training mechanism; the inference phase comprises target optimization and consistency verification for dual-directional generation.

The present disclosure also relates to a dual-directional content generation method based on a generative large language model, comprising following steps: collecting physiological signal data through a wearable device, acquiring a subjective symptom textual description provided by a user through an inputting device; integrates the collected physiological signal data and subjective symptom textual description into a structured health data; evaluating a health status of the user by analyzing the structured health data in real time through a deep sequence model; dynamically generating a content based on the evaluated results of health status, wherein the content comprises personalized treatment recommendations adapted to the user's needs and specialized diagnostic reports provided to a doctor; wherein the generating step comprises: adopting LORA fine-tuning technology to optimize the generated content, verifying and optimizing the generated content by a medical knowledge base and a regulatory rule base; and combining user's health status and medical resource condition, dynamically adjusting a frequency of content generation, and providing a personalized health report and a follow-up appointment planning.

Preferably, in the evaluating step, further comprises: detecting sudden changes using time series modeling; classifying the health status and judging whether current situation is an emergency situation; and outputting a health state classification information to serve as a trigger for generating subsequent content.

In certain embodiment, the generating step includes a training phase and an inference phase; the training phase is to optimize the generative large language model so as to generate the adapted content according to the needs of different target users; the inference phase is to classify and adjust the generated content to ensure matching needs of the target users.

Preferably, in the training phase, the data is classified and annotated per the target users to provide a direction for the training process; the parameters of the generative large language model is optimized through fine-tuning technique to enable generating the adapted content for a patient and a doctor; the training samples are extended through data augmentation to improve the model's ability to adapt to diverse scenarios; in the inference phase, the output is optimized based on the preliminary results of the generated content.

The present disclosure also relates to a computer readable storage medium storing a program that enables a computer to execute the dual-directional content generation method as described above.

The present disclosure breaks through the limitations of existing medical generation systems and provides an efficient, accurate, and intelligent solution in the field of medical information processing and health management. Specifically, the present disclosure constructs an intelligent and efficient dual-directional content generation system by integrating multi-modal data collection from wearable devices, deep sequence health status evaluation, dual-directional content generation from generative large language model, and dynamic content optimization technology. The present disclosure has the following technical advantages:
(1) Real-time response and personalized adaptation: Based on real-time health data and user feedback, the content generation logic is dynamically adjusted to provide patients with accurate and operative health management suggestions, and generate specialized diagnostic support content for doctors, which effectively improves the efficiency of medical collaboration;
(2) Legal compliance and ethical protection: The system adopts a medical knowledge base and a library of rules and regulations to automatically check and optimize the generated content to ensure legal compliance, and at the same time strictly protects users' privacy through data encryption and anonymization;
(3) High scalability and adaptability: the system is highly scalable and can fine-tune the generative large language model according to the needs of different medical scenarios, which is suitable for diversified applications such as chronic disease management, response to acute medical events, and multi-language medical scenarios, and provides a new solution for the development of medical intelligence;
(4) Innovative content generation technology: The system adopts LORA fine-tuning technology and data augmentation strategies, realizing efficient learning under the condition of a small amount of annotation data and improving the diversity and accuracy of the generated content;

### BRIEF DESCRIPTION OF THE DRAWINGS

FIG. 1 is a framework diagram of the dual-directional content generation system of the present disclosure.

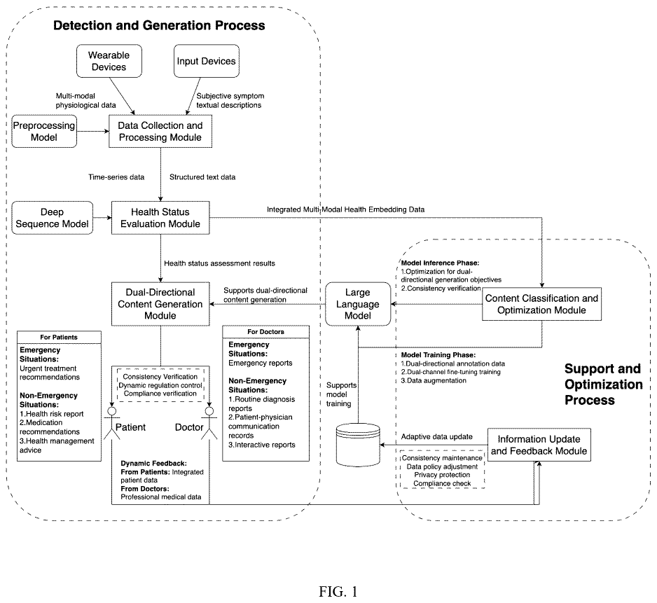

### DETAILED DESCRIPTION

In order to make the objects, technical solutions and advantages of the present disclosure more clearly understood, the embodiments of the present disclosure will be described in detail in the following with the accompanying drawings. It is to be noted that the embodiments and the features in the embodiments in the present disclosure may be combined with each other in any combination without conflict.

This disclosure proposes a dual-directional content generation system based on generative large language model, which could realize dual-directional content generation by collecting multi-modal physiological data of the user, such as physiological signals, user feedbacks, etc., combining the generative large language model with LORA (Low-Rank Adaptation) fine-tuning technology. As for the user's real-time health status, personalized health advices are generated, such as first aid measures, medication using guidance, etc. For doctors or health experts, specialized reports are generated based on the patient's health data and medical knowledge base, providing information such as clinical recommendations, lab test recommendations, and treatment options to facilitate doctors' decision-making. The system dynamically adjusts the style and depth of content generation based on user feedback and changes in health data to ensure the accuracy and real-time nature of the generated content, and uses LORA technology for fine-tuning the generative large language model to optimize model performance, efficiently learn on a small amount of annotated data and enhance the diversity and accuracy of the generated content.

A dual-directional content generation system based on a generative large language model according to the present disclosure, comprising the following subsystem:
(1) a detection and generation subsystem, including a multi-modal data collection and processing module, a health status evaluation module, and a dual-directional content generation module. These modules constitute the core workflow of the system and are responsible for the collection, evaluation, processing and generation of data flow.
(2) a support and optimization subsystem, including a content classification and optimization module and an information update and feedback module. These modules, as key parts for supporting the system to operate, could optimize the effect and real-time nature of the core process to ensure the accuracy and collaborative efficiency of content generation.

The present disclosure also relates to a dual-directional content generation method based on a generative large language model, comprising following steps: collecting physiological signal data through a wearable device, acquiring a subjective symptom textual description provided by a user through an inputting device; integrates the collected physiological signal data and subjective symptom textual description into a structured health data; evaluating a health status of the user by analyzing the structured health data in real time through a deep sequence model; dynamically generating a content based on the evaluated results of health status, wherein the content comprises personalized treatment recommendations adapted to the user's needs and specialized diagnostic reports provided to a doctor; wherein the generating step comprises: adopting LORA fine-tuning technology to optimize the generated content, verifying and optimizing the generated content by a medical knowledge base and a regulatory rule base; and combining user's health status and medical resource condition, dynamically adjusting a frequency of content generation, and providing a personalized health report and a follow-up appointment planning.

The present disclosure also relates to a computer readable storage medium storing a program that enables a computer to execute the dual-directional content generation method as described above.

### Multi-Modal Data Collection and Processing Module

The multi-modal data collection and processing module integrates the collected physiological signals data and subjective symptom textual description into a structured health data by means of denoising algorithms and time series modeling techniques. The module can collect multiple physiological signals in real time through wearable devices, including but not limited to heart rate, blood pressure, blood oxygen, body temperature, gait and exercise data, etc., and the frequency of data collection can be adjusted per requirement. The system converts the physiological signals into high-quality time-series data through techniques such as denoising algorithms, normalization processing and time-series resampling etc., Simultaneously, the system also generates text data from subjective symptom textual description input by the user and transforms it into structured input through natural language processing techniques. In the text data processing, the offending information is filtered to prevent it from entering the subsequent process. The generated time series data and structured text data are available for subsequent modules.

### Health Status Evaluation Module

The user's health data is analyzed in real-time to evaluate the user's health status through deep sequence models such as Bi-LSTM, Transformer, etc. For different health status assessment results, the health status evaluation module would trigger the content generation process. The system uses a dynamic threshold adjustment mechanism to automatically adjust the health status evaluation model in conjunction with real-time changes in user health data. Specifically, the system dynamically generates a comprehensive assessment result of the health status based on the health data embedding and user feedback, and determines whether it is an emergency or non-emergency situation.

### Dual-Directional Content Generation Module

The dual-directional content generation module dynamically generates a personalized treatment recommendations adapted to the user's needs and a specialized diagnostic reports for the doctor based on the health status assessment results. Based on the health status assessment results, the system triggers the dual-directional content generation module to generate personalized content for a patient and a doctor respectively. Personalized content can be divided into emergency situation generating content and non-emergency situation generating content.

Wherein, the emergency situation generating content for the patient is the system-generated emergent treatment recommendations, such as: “it is recommended to immediately call the emergency telephone”, “take alternative drugs and monitor blood pressure”. Emergency situation generating content for doctors is system-generated emergency reports, including the patient's current physical condition, such as heart rate, blood oxygen level and other abnormal indicators; emergency medication recommendations, such as temporary medication adjustment scheme; laboratory test recommendations, such as immediately arranging for an electrocardiogram. These contents are triggered at a high priority to ensure that the patient receives timely emergency measures and to provide rapid decision support to the doctor.

In non-emergency situation, the system dynamically generates the content based on health status assessment results and medical resource condition, and distinguishes between the specific needs of a patient and a doctor. For patients, the system can generate: health trend reports to provide visual graphs of health trends, such as changes in blood pressure and heart rate, supplemented with lifestyle recommendations, such as dietary adjustments and exercise planning; medication recommendations to generate daily medication plans or medication warnings based on the patient's medication record and assessment results, such as “remind to take antihypertensive medication on time”; health management advice, including routine precautions and immediate health goals in chronic disease management. For doctors, the system can generate: routine diagnosis reports to generate a comprehensive summary of health status, including long-term health trends such as blood pressure control trends, weight changes, analysis of the patient's treatment effects and potential health risks; medical recommendations and resource evaluations to provide recommendations for treatment adjustments based on the health evaluation, e.g., “recommend to adjust the medication dosage,” and dynamically recommend a follow-up time for patient based on the medical resource condition; interactive reports to generate interactive content for doctors, such as explanations of key data indicators, e.g., “the patient's blood pressure fluctuated less in the last month and was within the target range on average.

### Content Classification and Optimization Module

The content classification and optimization module adopts LORA fine-tuning technology to optimize the generated content under the condition of a small amount of annotation data, also can combine data augmentation strategies to expand the ability to adapt to diversified scenarios; through the medical knowledge base and the regulatory rule base to check and optimize the generated content to ensure the legal compliance of the generated content, and generate high-quality content that meets the needs of the target users for the patients and the doctors respectively. The generative large language model is fine-tuned using LORA technology to enhance the learning ability of the model under a small amount of annotation data. By categorizing the generated content, the system automatically adjusts the style of contents and depth of information according to patients or doctors. The content for patients is concise and highly operational, while the content for doctors contains detailed medical information and specialized advice. Simultaneously, the generated content must be in line with the user's comprehension ability and medical practice standards to avoid misleading a patient and a doctor. When the content generation is adjusted, it should be ensured that the references to the medical knowledge base are authoritative and legitimate.

### Information Update and Feedback Module

The system dynamically adjusts the frequency of content generation, taking into account the user's health status and medical resource condition, to provide personalized health reports and a follow-up appointment planning. The system continuously collects patient health data and feedback, and updates the model with new data on a regular basis or based on triggering conditions to optimize the content generation effect. The system calculates the frequency of content generation based on fluctuations in user health data, changes in medication, and other factors, and dynamically optimizes the health evaluation and content generation strategies to ensure the timeliness and accuracy of the generated content. Simultaneously, the system maintains the consistency of the patient and doctor information when generating content. For example, medication recommendations received by the patients are synchronized with medication adjustment recommendations in the doctor's diagnostic report to enhance information collaboration efficiency and output reliability. In addition, the data storage is ensured to comply with privacy protection and data compliance regulations, including data encrypted storage, anonymization and access rights control to avoid unauthorized use of user data.

A dual-directional content generation system based on a generative large language model according to the present disclosure, the workflow of which comprises:

### (1) Data Collection and Processing

Includes the following steps: use a wearable device to collect the user's physiological signals X in real time, including data such as heart rate, blood pressure, oxygen saturation, body temperature, etc., and the user provides subjective symptom textual description F via an input device; for the physiological signals data, denoising process is performed to ensure the quality of the data by cleaning up incomplete or abnormal signals; for the textual data, text vectorization is performed; and the pre-processed X′ and F′ are inputted to the health status evaluation module.

### (2) Health Status Evaluation and Classification

Integrate the pre-processed X′ and F′ into a multi-modal analysis model, where the trend data of physiological signals and the textual representations of subjective symptoms were used simultaneously as a basis for evaluation, to comprehensively analyze the data;

Use time-series modeling to detect the sudden changes, such as abnormal heart rates or sudden drops of blood oxygen;

Classify the health status and determine whether the current situation is an emergency situation, e.g., “emergency” or “non-emergency”;

Make the evaluation process be transparent and ensure that the output health state classification is clearly explained, e.g. “heart rate exceeds safe range”, and ultimately output the health state classification information that will serve as a trigger for the subsequent content generation.

### (3) Dual-Directional Content Generation

Trigger content generation process for different user types in different situations, based on the health status assessment results. All content generation is based on an authoritative medical knowledge base, which automatically avoids the potential misleading advice or the content that violates the medical practice code, enabling implicit compliance integration.

When an emergency situation is detected, the system triggers the dual-directional content generation. For the patient, generate the emergent treatment recommendations, e.g. “call emergency immediately” or “take alternative medication and monitor blood pressure”. For the doctor, generate emergency reports, e.g. “The patient has an abnormal heart rate and low oxygen saturation. Laboratory tests and an ECG are recommended”.

When the non-emergency situation is detected, the system dynamically adjusts the frequency of content generation based on the user's health status and the resources of the healthcare organization. For patients, generate the patient condition reports weekly, containing the health trends, medication and lifestyle recommendations. For doctors, generate the comprehensive health reports monthly or quarterly to track the development of the condition and evaluate the effectiveness of long-term treatment.

A dual-directional content generation system based on a generative large language model according to the present disclosure, the support and optimization process comprising:
(1) content classification and optimization, including a training phase and an inference phase;
The goal of the training phase is to optimize the generative large language model so as to generate the content adapted to the needs of different target users, such as patients or doctors. Specifically, it may include: classifying and labeling the data according to the target users to provide a direction for the training process; optimizing the parameters of the generative large language model through the fine-tuning technique to enable the generation of adapted content for a patient and a doctor; and expanding the training samples through data augmentation, e.g., by generating the transition data for different health states or by converting the multi-modal data into the natural language description, to enhance the model's ability to adapt to a variety of scenarios.

Specifically, for patients, the fine-tuned model generates the concise and easy-to-understand content that emphasizes practicality, such as medication recommendations and health advice. For doctors, the fine-tuned model generates detailed and professional content that supports the medical decision-making, such as the physiological data analysis and diagnostic advice. The input information of the training phase is the user history data, such as the content demand characteristics of a patient and a doctor, and the diverse training samples after data augmentation. The output information is the fine-tuned generative large language model, which is capable of distinguishing the user types and generating the high-quality content.

The goal of the inference phase is to classify and adjust the generated content to ensure matching the needs of the target users. Specifically, it can include: further optimizing the output based on the preliminary results of the generated content. For patients, the output should be concise and easy to understand, and should highlight the implementation, such as the medication recommendations. For doctors, the output should be detailed and logical, containing the necessary diagnostic information. The input information of the inference phase is the preliminary generated content from the “dual-directional content generation module”, and the output information is the final content that meets the needs of the target users, so as to improve the user experience and decision-making support effect.

Expressions that do not meet the needs of the target user or the medical norms, such as the non-specialized terminology or the polysemous and ambiguous cues, are automatically filtered during the content optimization of the training and inference phases.
(2) Information updating and feedback learning, including a training phase and an inference phase;
The goal of the training phase is to optimize the model performance by continuously collecting new data and updating the training dataset. Specifically, it includes: collecting the latest user feedback data, such as the patient's satisfaction with the content and the doctor's acceptance of the diagnostic content, and adding the new data to the model training process to dynamically adjust the generation policy and the parameters of the health status evaluation model. The input information of the training phase is the user's historical feedback data, and the output is the continuously optimized model, which is more adaptable to the user's needs.

The goal of the inference phase is to ensure that the system dynamically adjusts the health evaluation and content generation strategies based on the latest data and feedback during operation. Specifically, it includes:
dynamic updating: collecting the latest health data and feedback from users, and updating the system database on a regular basis or according to triggering conditions, so as to keep the evaluation model up-to-date;
policy adjustment: adjusting the policy of the health status evaluation model and the content generation module according to the new data, optimizing the timeliness and accuracy of the system's outputs; and
information consistency maintenance: ensuring that key information in the content generated for the patient and the doctor is consistent, such as medication advice synchronization. The inputs of the inference phase are the latest health data and feedback from the user and the current evaluation and generation policy of the system, and the outputs thereof are the dynamically adjusted health evaluation and the content generation policy, and the information-consistent generated content, which improves the efficiency and reliability of collaboration.

A dual-directional content generation system based on a generative large language model according to the present disclosure, a detection and generation process of which comprising the following steps:

### (1) Data Preprocessing and Feature Extraction

In the data preprocessing phase, the system transforms multi-modal data of the physiological signals and textual description into high-quality, structured inputs for the health evaluation and content generation modules.

Physiological signal denoising process:

X′=Filter(X)

Where, X is the original signal data; X′ is the denoised signal; Filter is the noise filtering algorithm.

Text vectorization process: using pre-trained language models such as BERT, GPT to generate text embeddings

F′=TextEmbed(F)

Where F′ is a vector representation of the text; TextEmbed is a vectorization method.

### (2) Health Status Evaluation and Classification

After the fusion of multi-modal data (the physiological signals and textual description), the user's health status is classified through comprehensive analysis and time series modeling, to provide clear explanations with key indicators to support the triggering of subsequent content generation modules.

Specifically, the algorithm for this step is as follows:

### \textcircled{1} Data Fusion

H=Fuse(X′,F′)

wherein, X′ is the physiological signal data, such as the denoised data, e.g. heart rate, blood pressure, blood oxygen, etc.; F′ is the textual vectorized data, the transformed vector representation of subjective symptom description; H is the multi-modal health embedding; and Fuse is a fusion function, which achieves the data integration by weighting or multi-modal network.

### \textcircled{2} Time Series Modeling

Use time-series models such as LSTM or Transformer to model trends in the physiological data and detect the sudden changes in physiological data, such as abnormal heart rates or sudden drops in blood oxygen.

Δ=TimeSeriesModel(X′)

Where, Δ is the detected abnormal physiological signal; TimeSeriesModel is a timing model function.

### \textcircled{3} Classification of Health Status

Input the fused health embedding H and the abnormal signal Δ, and use a classifier such as Softmax to determine the current health state:

P(c|H)=Softmax(W_{c}·H+b_{c})

Where, c is the health status classification (e.g., “emergency” or “non-emergency”); W_{c} and b_{c} are the weights and biases of the classifier.

### \textcircled{4} Interpretation of Health Status and Output of Key Indicators

Extract the significant features of the classifier, which are used to explain the health status:

Explanation=ExtractFeatures(H,Δ)

Where Explanation is a transparent interpretation: e.g. “heart rate exceeds the safe range and fluctuates too much”.

### (3) Dual-Directional Content Generation

Based on the health status assessment results, the system generates the personalized content for a patient and a doctor respectively, and adjusts the generation logic according to an emergency or non-emergency situation to ensure that the generated content is compliant and provides clear operability and decision support.

### \textcircled{1} Emergency Situation Content Generation

G_{P}=GenerateEmergencyPatient(H,C)

G_{D}=GenerateEmergencyDoctor(H,C)

Where C is a health emergency situation; H is a multi-modal health embedding; G_{P} is a patient content, e.g., first aid advice; and G_{P} is a doctor content, e.g., emergency diagnostic report. The generated content G includes a patient content G_{P} and a doctor content G_{P}.

Extract key entities from the generated content G, such as drug names, dosages, medical recommendations, and match these entities to the regulatory rule base [symbol] ={r₁, r₂, . . . , rₙ}

E=ExtractEntities(G)

S(E,rᵢ)=Sim(E,rᵢ)

Where E is a key entity in the generated content, r; is a regulation rule, and Sim is a semantic similarity function.

If the content does not meet the requirements of the regulation:

G′=Correct(G,R)

Where G′ is an amended content to ensure compliance.

### 2) Non-Emergency Situation Content Generation

Dynamically adjust the frequency of report generation based on the user's health status and medical resource condition.

f=AdjustFrequency(H,rₘ)

Where H is a fused health embedding, which reflects a combination of recent physiological signals and feedback data; rₘ is a healthcare resource profile, such as doctor availability or schedule of healthcare institution; and f is the generation frequency.

Content generation is then performed based on H, C and f

G_{P}=GenerateNonEmergencyPatient(H,C,f)

G_{D}=GenerateNonEmergencyDoctor(H,C,f)

Where, G_{P} is a patient content, including generating health trend reports and providing medication recommendations and lifestyle advice; G_{P} is a doctor content, including generating routine diagnosis reports, analyzing long-term trends, and suggesting treatment adjustments and a follow-up appointment planning.

By extracting key information, comparing consistency and dynamically correcting, the final output is a consistent content for patient and doctor. Specifically, it includes:
key information extracting:

K_{P}=ExtractKeyInfo(G_{P})

K_{D}=ExtractKeyInfo(G_{D})

Where K_{P} is the key information in the patient content; and K_{D} is the key information in the doctor content.
information matching:

ConsistencyScore=Compare(K_{P},K_{D})

Where, ConsistencyScore is the consistency score, which measures the matching degree between the patient content and the doctor content.

Consistency verification and correction

$${G}_{P}^{′} , {G}_{D}^{′} = \{ \begin{matrix}{G}_{P} , {G}_{D} & if ConsistencyScore \geq Threshold \\ Correct ( {G}_{P} , {G}_{D} ) & if ConsistencyScore < Threshold\end{matrix}$$

When ConsistencyScore falls below a threshold, the patient and doctor contents G_{P}, G_{D} are dynamically corrected to generate consistent content G′_{P}, G′_{D} to ensure key information is consistent.

A dual-directional content generation system based on a generative large language model according to the present disclosure has a support and optimization process comprising content classification and generation optimization, information updating and feedback learning, wherein the content classification and generation optimization specifically comprises:

### Training Phase

(1) Low-Rank matrix LORA fine-tuning for different objects

Model parameter update equations: W′=W+A·B

Where, W is the original model weights; A and B are the trainable low-rank matrices; W′ is the optimized weights.

The fine-tuning process is optimized for the different goals of patient and doctor generated content respectively. For patients: to optimize the simplicity and operability of the generated emergency recommendations and health management reports. For doctors: to optimize the professionalization and the details of the generated diagnostic reports and treatment recommendations.

(2) Dual-channel training mechanism, where the model introduces a dual-channel training policy to design the annotation datasets for a patient and a doctor respectively.

For patient channel, includes the annotation data such as health trend description, lifestyle recommendations, medication recommendations, and more;

For doctor channel, includes the specialized data on the interpretation of signs and indicators, the clinical recommendations, and the laboratory test protocols, etc.

During the dual-channel training, the LORA technique allows the model to perform a domain-specific fine-tuning based on the shared parameters:

Patient-generated content optimization:

G_{P}=LORA(W,D_{P})

Where D_{P} is the patient labeling data.

Doctor-generated content optimization:

G_{D}=LORA(W,D_{D})

where D_{D} is the doctor labeling data.

(3) Data augmentation, including interpolation, modal transition, and cross-enhancement.

First, a transition data for different health states is generated by interpolation:

X_{aug}=Interpolate(X_{low},X_{high})

where X_{low} and X_{high} are the boundary values for the health state data and X_{aug} is the generated transition data.

Then, the multi-modal data is converted into the natural language description to enhance the model's adaptability to diverse scenarios:

T=Convert(X′)

where Convert is the conversion function that transforms the signals of the physiological signs into the natural language description.

Finally, the partial data sharing between the patient and doctor channels is performed for cross-enhancement to ensure the logical consistency of the dual-directionally generated content.

### Inference Phase

(1) Goal optimization for the Dual-directional generation:

The model optimizes the dual-directional fitness of the generated content by the following formula:

P(G_{O}|H,T)=Softmax(W′·G+b)

Where G_{O} is the generated optimized content, H is the input fused health data embedding, T denotes the generation target (patient or doctor), G is the initial content generated after channeling, and b is a bias to adjust the final score or probability distribution of the generated content.

(2) Consistency verification:

Calibrate the content through the medical knowledge base:

Valid(G)=CheckCompliance(G,Knowledge)

WhereCheckCompliance is the content compliance verification function and Knowledge is the medical knowledge base.

Among them, information updating and feedback learning is specifically designed to continuously optimize the model performance and content generation strategies by dynamically collecting user data and feedback, and simultaneously embed privacy protection and compliance check mechanisms. Included:

### Training Phase

Update the model with the latest user feedback to improve the accuracy and practicability of the generated content.

### Inference Phase

Dynamically adjust strategies and content generation at runtime to improve the timeliness and reliability of the system.

(1) Dynamic data updating:

DB′=UpdateDatabase(DB,NewData)

Collect the latest health data and feedback information NewData from users and update the database DB to get a new database DB′.

(2) Strategy Adjustment:

Strategy′=AdjustStrategy(DB′)

Dynamically adjusting the health evaluation and content generation strategies based on the updated database DB′.

(3) Maintenance of information consistency: Ensure synchronization of key information in patient-generated content and doctor-generated content.

### Embedding Privacy and Compliance

(1) Privacy protection: User data is anonymized and encrypted to ensure that the privacy is not compromised.

(2) Compliance check: A compliance checking mechanism is embedded to ensure legal and transparent use of data and avoid potential legal risks.

This disclosure successfully achieves dual-directional generation of health content through a generative large language model combined with a dynamic optimization strategies. which could provide the personalized health advice for patients and generate the in-depth diagnostic reports for doctors. With this dual-directional generation framework, the system is able to respond in real-time to the changes in the user's health status, improving the collaboration efficiency and content accuracy in medical scenarios.

What is claimed:

1. A dual-directional content generation system based on a generative large language model, comprising a computer configured to execute a program stored in a computer-readable storage medium readable by the computer to implement a detection and generation subsystem and a support and optimization subsystem, wherein the detection and generation subsystem comprises a multi-modal data collection and processing module, a health status evaluation module and a dual-directional content generation module, the support and optimization subsystem comprises a content classification and optimization module and an information update and feedback module;
wherein, the multi-modal data collection and processing module integrates a collected physiological signal data and a subjective symptom textual description into a structured health data by means of a denoising algorithm and a time series modeling;
the health status evaluation module evaluates a health status of a user by analyzing the structured health data in real time through a deep sequence model;
the dual-directional content generation module dynamically generates personalized treatment recommendations adapted to user's needs and specialized diagnostic reports provided to a doctor based on health status assessment results;
the content classification and optimization module adopts LORA fine-tuning technology to optimize the generated content, verifies and optimizes the generated content by a medical knowledge base and a regulatory rule base;
the information update and feedback module dynamically adjusts a frequency of content generation based on the health status of the user and a medical resource condition, and provides personalized health reports and a follow-up appointment planning based on the medical resource condition.

2. The dual-directional content generation system according to claim 1, wherein the content generated by the dual-directional content generation module comprises an emergency situation generated content and a non-emergency situation generated content.

3. The dual-directional content generation system according to claim 1, wherein the content classification and optimization module includes a training phase and an inference phase; the training phase is to optimize the generative large language model so as to generate an adapted content per different target users; the inference phase is to classify and adjust the generated content to ensure matching the needs of the target users.

4. The dual-directional content generation system according to claim 3, wherein in the training phase, the data is classified and annotated per the target users to provide a direction for the training process; parameters of the generative large language model is optimized through fine-tuning technique to enable generating the adapted content for a patient and a doctor; training samples are extended through data augmentation to improve the model's ability to adapt to diverse scenarios.

5. The dual-directional content generation system according to claim 4, wherein in the inference phase, the output is optimized based on the preliminary results of the generated content; the input information of the inference phase is the preliminary generated content of the dual-directional content generation module, and the output information is the final content that meets the needs of the target user.

6. The dual-directional content generation system according to claim 1, wherein the information update and feedback module comprises a training phase and an inference phase, the training phase is to add a new data to model training process by continuously collecting new data, dynamically adjust generation strategies and the parameters of the health status evaluation model, continuously update training dataset and optimize model performance;
the inference phase is to collect latest health data and feedback information from the user and update the system database periodically or per triggering conditions to keep the evaluation model up-to-date; and to adjust the strategies of the health status evaluation model and the content generation module per the new data to optimize the output of the system.

7. The dual-directional content generation system according to claim 1, wherein workflow of the system comprises:
(1) collecting and processing the data
wherein the user's physiological signalsX and the subjective symptom textual descriptionF provided by the user through an input device are collected in real-time; for the physiological signals data, a denoising process is performed to ensure the quality of the data by cleaning up the incomplete or abnormal signals; for the textual data, the text vectorization processing is performed; the pre-processed physiological signalsX and subjective symptom textual descriptionF′ are inputted to the health status evaluation module;
(2) evaluating and classifying health status
wherein pre-processed physiological signals X″ and subjective symptom textual description F″ are integrated into a multi-modal analysis model, where trend data of the physiological signals and textual representations of subjective symptoms are used as a basis for evaluation to comprehensively analyzing the data;
(3) generating dual-directional content
wherein content generation process for different users in different situations is triggered based on health status assessment results.

8. The dual-directional content generation system according to claim 7, wherein evaluating and classifying health status comprises:
detecting sudden changes using time series modeling;
classifying health status and determining whether current situation is an emergency situation, and ultimately output health state classification information that serves as a trigger for generating subsequent content.

9. The dual-directional content generation system according to claim 1, wherein the content generation process comprises the following steps:
(1) in data preprocessing phase, structuring multi-modal data of physiological signals and textual description, using as input data for the health status evaluation module and the dual-directional content generation module;
(2) evaluating and classifying user's health status by comprehensive analyzing and time series modeling the input data, for triggering of subsequent content generation modules;
(3) based on health status assessment results, generating a personalized content for a patient and a doctor respectively, and adjusting generation logic according to an emergency or a non-emergency situation to ensure that generated content is compliant and provides treatment recommendation.

10. The dual-directional content generation system according to claim 1, wherein content classification and generation optimization process comprises a training phase and an inference phase, the training phase comprises low rank matrix LORA fine-tuning for different objects and annotates dataset for a patient and a doctor respectively by a two-channel training mechanism; the inference phase comprises target optimization and consistency verification for dual-directional generation.

11. A dual-directional content generation method based on a generative large language model, comprising following steps:
collecting physiological signal data through a wearable device, acquiring a subjective symptom textual description provided by a user through an inputting device;
integrates the collected physiological signal data and subjective symptom textual description into a structured health data;
evaluating a health status of the user by analyzing the structured health data in real time through a deep sequence model;
dynamically generating a content based on the evaluated results of health status, wherein the content comprises personalized treatment recommendations adapted to the user's needs and specialized diagnostic reports provided to a doctor;
wherein the generating step further comprises:
adopting LORA fine-tuning technology to optimize the generated content, verifying and optimizing the generated content by a medical knowledge base and a regulatory rule base; and
dynamically adjusting a frequency of content generation based on the health status of the user and a medical resource condition, and providing a personalized health report and a follow-up appointment planning based on the medical resource condition.

12. The dual-directional content generation method according to claim 11, wherein the evaluating step further comprises:
detecting sudden changes using time series modeling;
classifying the health status and judging whether current situation is an emergency situation; and outputting a health state classification information to serve as a trigger for generating subsequent content.

13. The dual-directional content generation method according to claim 11, wherein the generating step includes a training phase and an inference phase; the training phase is to optimize the generative large language model so as to generate the adapted content according to the needs of different target users; the inference phase is to classify and adjust the generated content to ensure matching needs of the target users.

14. The dual-directional content generation method according to claim 13, wherein in the training phase, the data is classified and annotated per the target users to provide a direction for the training process; the parameters of the generative large language model is optimized through fine-tuning technique to enable generating the adapted content for a patient and a doctor; the training samples are extended through data augmentation to improve the model's ability to adapt to diverse scenarios;
in the inference phase, the output is optimized based on the preliminary results of the generated content.

15. A non-transitory computer readable storage medium storing a program that enables a computer to execute the dual-directional content generation method according to claim 11.

16. The dual-directional content generation system according to claim 1, wherein the system extracts key information from the personalized treatment recommendations and key information from the specialized diagnostic reports, computes a consistency score by comparing the extracted key information, and corrects at least one of the personalized treatment recommendations and the specialized diagnostic reports when the consistency score is below a threshold.

17. The dual-directional content generation method according to claim 11, further comprising: extracting key information from the personalized treatment recommendations and key information from the specialized diagnostic reports, computing a consistency score by comparing the extracted key information, and correcting at least one of the personalized treatment recommendations and the specialized diagnostic reports when the consistency score is below a threshold.
